(19) United States
(12) Patent Application Publication
(10) Pub. No.: US 2019/0083294 A1
(43) Pub. Date: Mar. 21, 2019

(54) STAND FOR COLLECTING FECAL MATTER

(71) Applicant: James Paul Dustin, Jacksonville, FL (US)

(72) Inventor: James Paul Dustin, Jacksonville, FL (US)

(21) Appl. No.: 16/128,858

(22) Filed: Sep. 12, 2018

Related U.S. Application Data
(60) Provisional application No. 62/558,934, filed on Sep. 15, 2017.

(51) Int. Cl. A61F 5/445

(52) U.S. Cl. CPC A61F 5/445; A61F 2005/4495

(57) ABSTRACT

A collection vessel for fecal matter. The collection vessel includes a base configured to support the collection vessel in an upright position. A hollow tube has a bottom end and a top end. The bottom end is coupled to the base and the top end includes a top edge forming an opening. At least one cup includes an upper rim and a body. The cup body fits within the opening and the upper cup rim rests against the top edge of the hollow tube.

### CROSS-REFERENCE TO RELATED APPLICATION

This application claims the benefit of priority of U.S. provisional application No. 62/558,934, filed Sep. 15, 2017, the contents of which are herein incorporated by reference.

### BACKGROUND OF THE INVENTION

The present invention relates to assisting ostomy gastrointestinal (GI) patients and, more particularly, to a stand for ostomy patients to empty their fecal waste temporarily, in a 2-step process.

People who have an external ostomy pouch attached to their body with adhesive around a stoma (an alternative for bowel movement caused by varied diseases) must empty waste material from such a pouch into a toilet. From a standing position, it can cause splashing and may even miss the target. From a kneeling position, the person is on the floor, which can cause stress to the knees. Sitting on the toilet is awkward, as the person must either sit side-saddle, or empty between the legs. This is particularly hard, because there is some cleaning of the bottom of the ostomy pouch before closing it back up. Reaching for materials and maneuvering the pouch is difficult. It often becomes extra difficult with overweight individuals.

A current ostomy product employs a rigid sack made of plastic held around a person's waist, with a liner, for pouring waste into. The liner can be bio degradable and flushed. While this is a good solution for travel and out of the home, it is not a convenient solution for the home.

As can be seen, there is a need for an improved, comfortable, and reusable ostomy-emptying utility product for GI ostomy patients.

### SUMMARY OF THE INVENTION

In one aspect of the present invention, a collection vessel for fecal matter comprises: a base configured to support the collection vessel in an upright position; a hollow tube comprising a bottom end and a top end, wherein the bottom end is coupled to the base and the top end comprises a top edge forming an opening; and at least one cup comprising an upper rim and a body, wherein the body fits within the opening and the upper rim rests against the top edge of the hollow tube.

In another aspect of the present invention, a method of disposing of fecal matter comprises the steps of: providing a collection vessel comprising: a base configured to support the collection vessel in an upright position; a hollow tube comprising a bottom end and a top end, wherein the bottom end is coupled to the base and the top end comprises a top edge forming an opening; and at least one cup comprising an upper rim and a body, wherein the body fits within the opening and the upper rim rests against the top edge of the hollow tube; and emptying fecal matter from an ostomy pouch into the at least one cup.

These and other features, aspects and advantages of the present invention will become better understood with reference to the following drawings, description and claims.

### BRIEF DESCRIPTION OF THE DRAWINGS

FIG. 1 is a perspective view of an embodiment of the present invention;

FIG. 2 is a front elevation view of an embodiment of the present invention;

FIG. 3 is a detail cross-sectional view taken along line 3-3 of FIG. 1;

FIG. 4 is detail cross-sectional view taken along line 4-4 in FIG. 1; and

FIG. 4a is a detail cross-sectional view of an embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention is a cup holder stand that allows the individual to pour the ostomy pouch waste (fecal material) into a cup, rather than trying to pour directly into the toilet. The cup holder is physically separate from a bathroom vanity or the toilet itself. The person can empty the bag waste into the cup at waist height, clean the pouch opening edge, and close the bag in a comfortable position. Advantageously, this two-step procedure using the cup stand, gives the user confidence of lastly pouring the contents directly into the toilet, and the precision to do this procedure while standing. Many patients struggle with directly emptying fecal matter into a toilet from a pouch. The present invention includes a two-step procedure with a separate cup and holder to aid in waste removal. The cup stand is also always ready and easy to move with handles.

The improvement for at-home use is that the stand is stationary and separate from the user and not hanging from the user's body like other devices. This allows the user or a caregiver, to have maximum control without the hindrances of a strap and further removes the device from the person, thereby avoiding the smell. The stand simply holds the cup, which after the waste is emptied into, is poured into the toilet.

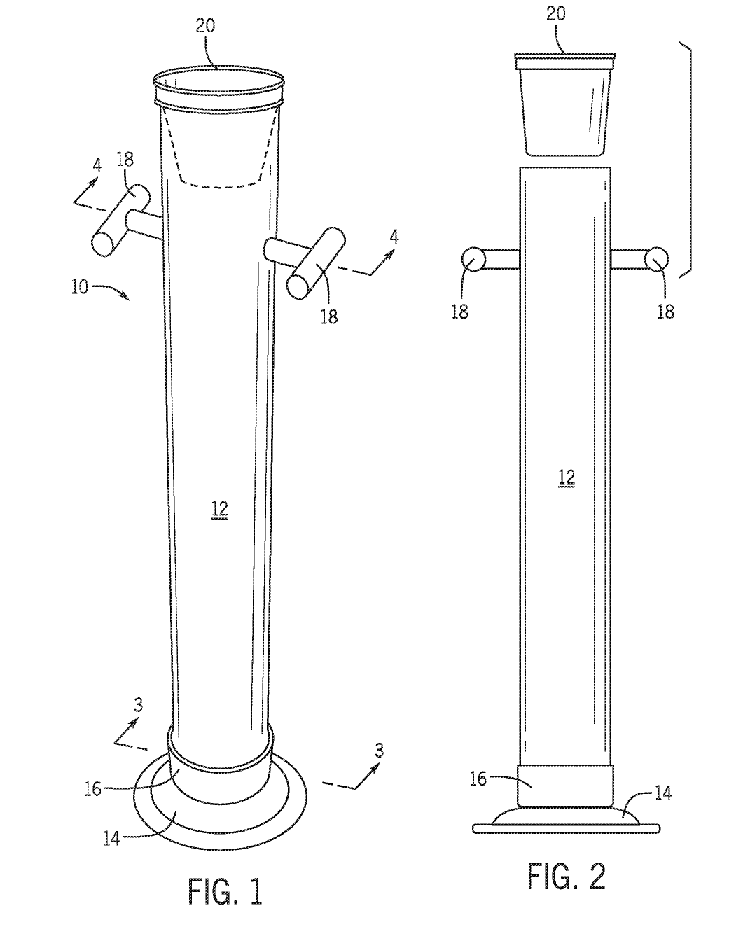

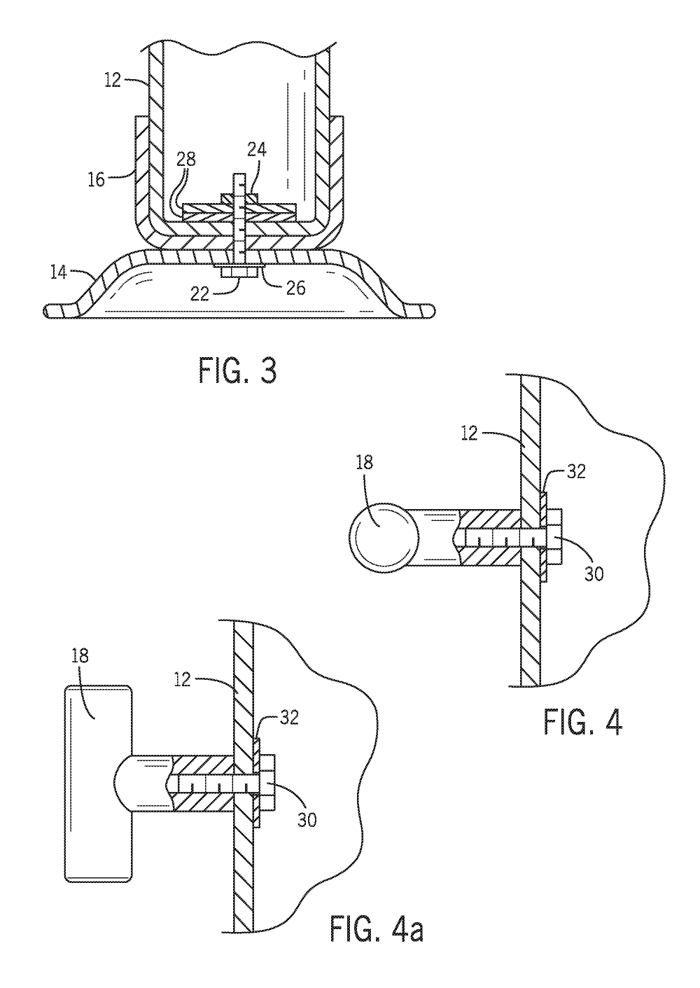

Referring to FIGS. 1 through 4a, the present invention includes a collection vessel for fecal matter 10. The collection vessel 10 includes a base 14 configured to support the collection vessel 10 in an upright position. The present invention further includes a hollow tube 12 having a bottom end and a top end. The bottom end is coupled to the base 14 and the top end includes a top edge forming an opening. At least one cup 20 includes an upper rim and a body. The cup body fits within the opening and the upper cup rim rests against the top edge of the hollow tube 12. The at least one cup 20 may include a plurality of cups 20, allowing the cup 20 in use to be washed and another cup 20 ready for future use.

The base 14 may include an upside-down dish shape with a flange extending radially from the bottom rim. Therefore, the base 14 includes a wide profile, which prevents the collection vessel 10 from tipping over. In certain embodiments, a weight 28 is coupled to at least one of the base 14 and the bottom end of the hollow tube 12. The weight 28 may also prevent the collection vessel 10 from tipping over.

In certain embodiments, the present invention may include a support 16 coupling the hollow tube 12 to the base 14. In such embodiments, the present invention may further include a bolt 22, a nut 24, and a washer 26. The bolt 22 runs through aligned openings of the washer 26, the base 14, the support 16, the hollow tube 12, and the weight 28. The nut 24 is secured to the end of the bolt 22, holding the base 14, the support 16, the hollow tube 12, and the weight 28 together.

The present invention may further include handles 18 coupled to opposing sides of the hollow tube 12. The handles 18 may be in the shape of a “T” with the top of the “T” shape used as the outermost handle. Each of the handles 18 receives a threaded bolt 30. The bolt 30 extends through an opening of the hollow tube 12 and a washer 32. The handles 18 are then secured to the hollow tube 12 by tightening bolt 30. The handles 18 allow a user to grasp and reposition the collection vessel 10.

A method of disposing of fecal matter comprising the steps of: providing a collection vessel 10 described above; and emptying fecal matter from an ostomy pouch into the at least one cup 20. The method may further include the step of emptying the fecal matter from the at least one cup 20 into a toilet and flushing the toilet.

In one embodiment, the primary function of the invention is to securely hold the 1-quart cup in a stand at the same height as the ostomy pouch. The external ostomy pouch apparatus attaches to the body, at the stoma. A stoma is an opening on the surface of the abdomen which has been surgically created to divert the flow of feces or urine, usually at the identical height as a belly button, but off to either the left or right side.

Therefore, the invention secures the cup at that same height to make it easy to pour the waste contents into the cup. The primary function of the invention is to provide a stable and physically separate cup holder at a comfortable height that simplifies the 2-step process of 1) emptying waste into the cup and 2) further emptying this waste into the toilet from a lower position (mere inches).

To provide stability and the height for the cup, an approximately 4-inch-wide tube is utilized for the stand, which accepts the cup into the tube easily, but also remains perfectly rigid, thus creating a stable line of gravity to the base.

In one embodiment, to aid in easily moving the stand into the optimal position in the bathroom or toilet area, two handles are attached to the side of the stand. The handles can also act as a handy place to hang a small squirt bottle containing a diluted bleach solution for sanitary clean up.

In one embodiment, to make the invention, starting with the base unit: a plate is aligned convex side up and on center with drain cap, which is hollow-side up. Approximately a 5/16-diameter hole is drilled through both the drain cap and plate. The pieces are attached together with a 1.25 hex bolt, a ¾ inch washer next to the plate and received on the inside by a large 3 in. heavy galvanized dock washer, another ¾ inch washer and a 5/16 hex nut, completing the base unit. The tube is made of readily available white PVC sewer pipe, from which a ten-foot standard length is cut into 4 tube sections of 30 in. each. The prepared tube is inserted into the base unit and cemented or pressure fitted using packaging tape. From the top of the vertical tube, two holes are drilled 4 inches down from the top edge and opposite each other. Align each handle to the ⅛ inch hole to receive a SAE #8 washer and 4 mm hex bolt on the inside. Complete this for both handles. The final component is the 1-qt. plastic cup inserted into the top of the tube/column.

At this time, there may be an additional safety features added. That feature would be to glue a cup to the inside top of the tube and cut it off flush. This feature would guarantee that even if the user was in dim light or in a hurry and had forgotten to place a cup inside the tube, they could not empty into a hollow tube causing a mess. A floating cup would fit inside the tube as normal, with a safety cup permanently in place.

In one embodiment, the base and the 4-inch tube bring the stand up to waist height. It is to be understood that various suitable mechanisms can be added to adjust the device to a comfortable height, including pre-specified sizes.

In one embodiment, a collar can slip over the top of the tube and rest on the handles. This collar acts as a tray for putting cleaning materials such as rolled up toilet paper, wet naps, and a place to set the ostomy bag clip (if they use that type), while the bag is being prepared for emptying into the cup.

In one embodiment of use, the cup holding stand is ready to use when the patient enters the bathroom, provided there is a 1 QT. cup in place at the top of the stand. There could be several cups stacked upon each other to provide a slightly higher position. To solve the problem of awkwardly sitting on the toilet or standing to try to empty their ostomy bag from waist height into the toilet (causing splashing or missed trajectory), they simply use the cup holding stand. They unclip the bottom of the Ostomy pouch (some have a built-in Velcro latch, others use a clip). The bottom of the pouch is held in an upright position to keep the waste contents from spilling out. By using the cup stand, the patient pours the fecal waste contents into the quart cup, with precision and confidence. The waste can sit in the cup until the user is finished cleaning up and re-closing the pouch, or the cup can be picked up and emptied into the toilet immediately. The cup comes with a sealable lid to facilitate safe transport of the cup for caregivers from one room to another, or to cover the cup to reduce odor. The user has complete control over this 2-step process and ends the worry of hitting the toilet, spilling on their leg, or any other scenario of emptying the pouch directly into the toilet.

In summary, the user enters bathroom, unclips their pouch, and prepares for emptying. Instead of emptying into the toilet, they empty at waist height into a 1 Qt. cup that sits securely in a stand. At their own timing, they can then pick up the cup and pour the contents into the toilet without splashing or missing the target. They can then spritz the cup with a diluted bleach solution from a spray bottle, or squirt bottle to hygienically prepare the cup, placing it back into the stand for the next usage.

The invention is designed to hold a 1 Qt. plastic cup at approximately 30 inches high. Other embodiments, however, could include, but are not limited to: a candle holder, an ashtray, a drink holder, a plant holder, a smart phone holder or even rolled magazine. However, the invention is made to be in a bathroom to aid with waste removal for ostomy patients. It is to be understood that the size of the cup can be of any suitable size. Also, the sizes of the components can be any suitable sizes.

It should be understood, of course, that the foregoing relates to exemplary embodiments of the invention and that modifications may be made without departing from the spirit and scope of the invention as set forth in the following claims.

What is claimed is:

1. A collection vessel for fecal matter comprising:
a base configured to support the collection vessel in an upright position;
a hollow tube comprising a bottom end and a top end, wherein the bottom end is coupled to the base and the top end comprises a top edge forming an opening; and
at least one cup comprising an upper rim and a body, wherein the body fits within the opening and the upper rim rests against the top edge of the hollow tube.

2. The collection vessel for fecal matter of claim 1, further comprising a weight coupled to at least one of the base and the bottom end of the hollow tube.

3. The collection vessel for fecal matter of claim 2, further comprising a support coupling the hollow tube to the base.

4. The collection vessel for fecal matter of claim 3, further comprising a bolt and a nut, wherein the bolt runs through aligned openings of the base, the support, the hollow tube and the weight, and the nut is secured to the end of the bolt, holding the base, the support, the hollow tube and the weight together.

5. The collection vessel for fecal matter of claim 1, further comprising handles coupled to opposing sides of the hollow tube.

6. The collection vessel for fecal matter of claim 5, wherein each of the handles receives a threaded bolt, wherein the bolt extends through an opening of the hollow tube through a washer and is tightened.

7. The collection vessel for fecal matter of claim 1, wherein the at least one cup comprises a plurality of cups stacked together.

8. A method of disposing of fecal matter comprising the steps of:
providing a collection vessel comprising:
a base configured to support the collection vessel in an upright position;
a hollow tube comprising a bottom end and a top end, wherein the bottom end is coupled to the base and the top end comprises a top edge forming an opening; and
at least one cup comprising an upper rim and a body, wherein the body fits within the opening and the upper rim rests against the top edge of the hollow tube; and
emptying fecal matter from an ostomy pouch into the at least one cup.

9. The method of claim 8, further comprising the step of:
emptying the fecal matter from the at least one cup into a toilet and flushing the toilet.

10. The method of claim 8, wherein the collection vessel further comprises a weight coupled to at least one of the base and the bottom end of the hollow tube.
